(19) United States
(12) Patent Application Publication
(10) Pub. No.: US 2023/0144313 A1
(43) Pub. Date: May 11, 2023

(54) APPARATUS AND METHOD FOR FEMALE URINE SAMPLE COLLECTION

(71) Applicant: Limor GROSZ, Jordan Valley (IL)

(72) Inventor: Limor GROSZ, Jordan Valley (IL)

(21) Appl. No.: 17/911,686

(22) PCT Filed: Dec. 7, 2021
(86) PCT No.: PCT/IL2021/051453

§ 371 Date: Sep. 18, 2022

Related U.S. Application Data
(60) Provisional application No. 63/140,867, filed on Jan. 24, 2021.

(51) Int. Cl. B01L 3/00; A61B 10/00

(52) U.S. Cl. CPC B01L 3/508; A61B 10/007; B01L 2200/087; B01L 2200/141; B01L 2200/085; B01L 2300/041; B01L 2400/049; B01L 2400/06

(57) ABSTRACT

The present invention provides systems, methods and apparatus for enabling a female user to hygienically collect a urine sample, the apparatus includes a container including a urine collection body for receiving a urine sample from the female user with and a separator mechanism for separating a first volume of urine of the urine sample to a first urine storage element and a second volume of urine of the urine sample to a second urine storage element, a lid for closing the container after the urine sample has been received and optionally a handle for holding the container.

### RELATED APPLICATION

This application claims the benefit of priority under 35 USC § 119(e) of U.S. Provisional Patent Application No. 63/140,867 filed 24 Jan. 2020, the contents of which are incorporated herein by reference in their entirety.

### FIELD OF THE INVENTION

The present invention, relates in general to systems and methods for bodily fluid sample collection, and more specifically to apparatus and methods for collecting urine from female patients.

### BACKGROUND OF THE INVENTION

There remains an unmet need to provide improved ergonomic apparatus and methods for collecting urine samples from female patients. Some background art includes U.S. Pat. No. 3,340,876, which describes a pediatric urine collector with adhesive coated attaching pad.

U.S. Pat. No. 8,043,230 discloses a urine specimen collection device includes a body adapted to support a specimen collector. The device includes a pull tab movably connected to the body, the pull tab being movable between a first position and a second position. The urine selectively flows through the body and into the specimen collector when the pull tab is generally positioned in the second position and urine flows through the body and into a diverter tube when the pull tab is generally positioned in the first position.

U.S. patent Ser. No. 10/335,121 describes a receiver for urine, whether for taking sample or disposing thereof, includes an open-topped of saddle shape which approximates the configuration of thighs, labia or other convenient zone.

Japanese Patent no. JP4007999B2 discloses a female urine collector useful for collecting urine from a woman at home, a hospital, a clinic, and the like.

Chinese Utility Model no. CN201101657Y describes a utility model, which relates to an all-posture urine collection set for female patients, which comprises a urine container. The upper end of the urine container is provided with a corrugated anastomosis interface to be used as a urine holding port, and the corrugated anastomosis interface is of oval and is in shape of a circular arc at the lateral view. The corrugated anastomosis interface and the main body of the urine container are in shape of an inverted J to form a bending angle of 120 to 150 degrees, and an inverted V-shaped handle is arranged on the top of the urine container, namely the cross position of the back of the corrugated anastomosis interface.

A handgrip is made at the middle lower part of the urine container. all-posture urine collection set for female patients has the beneficial effects that because the corrugated anastomosis interface is of oval and is in shape of the circular arc at the lateral view, and the corrugated anastomosis interface and the main body of the urine container are in shape of the inverted J to form the bending angle of 120 to 150 degrees, no matter in the situations of prone position, sitting position, standing position and squatting position, patients can use through self-handhold, and nursing staff can hold the handgrip to help urine holding. The utility model is accorded with psychological, and the use is convenient and the measurement is easy.

In view of the limited background art, there still remains an unmet need to provide improved ergonomic apparatus and methods for collecting sterile urine samples from human female subjects.

### SUMMARY OF THE INVENTION

According to an aspect of some embodiments of the invention, there is provided a device enabling a female user to collect a urine sample, the device including: a sample compartment for collecting a sample of the urine; and a first stream separator for separating a first stream of urine from the sample of the urine and directed the sample of the urine to the sample compartment.

According to some embodiments of the invention, the device further includes a lid for closing the sample compartment after the urine sample has been received.

According to some embodiments of the invention, the lid is movable connected to the device such that when open the lid projects from an anterior edge of the device and is configured for use as a handle holding the device in a position to receive a urine stream.

According to some embodiments of the invention, the sample compartment and the first stream separator are included in a urine collection body having a saddle shape.

According to some embodiments of the invention, the body includes a wider collection area and a narrower drainage area.

According to some embodiments of the invention, the sample compartment is expandable.

According to some embodiments of the invention, the sample compartment includes an accordion-like expandable wall.

According to some embodiments of the invention, the device further includes a tactile position marker.

According to some embodiments of the invention, the collection body includes a lip configured to contract the body of the user and wherein the tactile position marker includes a protrusion from the lip.

According to some embodiments of the invention, the body has a wide proximal end configured to face anteriorly during use and a distal end configured to fit between legs of the user and face posteriorly and wherein the tactile position marker is disposed at a distal portion of the body.

According to some embodiments of the invention, the first stream separator includes a first stream compartment for storing the first stream.

According to some embodiments of the invention, the first stream compartment includes a cut off valve for closing the first stream compartment after receiving a predetermined quantity of fluid.

According to some embodiments of the invention, the device further includes a needle port opening into the sample compartment for withdrawing at least a part of the urine sample under vacuum.

According to some embodiments of the invention, the device further includes a tube receiving port for receiving at least one of a vial and a tube.

According to some embodiments of the invention, the device further includes at least one of: an opening tab; a dipstick port; a connector to lab apparatus (in vitro connection), a stabilizer axis; a vacuum mechanism; a sanitary sticker; and a position marker.

According to an aspect of some embodiments of the invention, there is provided a method collecting a urine sample from a female user, the method including: providing a device including a sample compartment and a first stream separator; directing a stream of urine from the user into the device; separating a first part of the stream of urine and directing it away form the sample compartment and collecting a volume of the urine stream subsequent to the first stream into the sample compartment.

According to some embodiments of the invention, the method further includes retaining a sterility of the volume of urine.

According to some embodiments of the invention, the method further includes transferring the volume directly from the sample compartment to a sample tube.

According to some embodiments of the invention, the method is performed by the female user.

According to some embodiments of the invention, the method further includes: opening a lid of the device to an open position; holding the device with the lid in the open position during the directing; and closing the lid after the directing to seal the sample compartment.

According to an aspect of some embodiments of the invention, there is provided a device for collecting a urine sample from a female subject including: a saddle-shaped body including an expandable sample compartment configured expand to receive a urine sample; a vacuum extractor port opening to the sample compartment and configured for draining the urine sample directly from the sample compartment to a vacuum sampler.

According to some embodiments of the invention, the saddle shaped container includes a wider anterior collection portion and a narrow posterior drainage portion.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1F are block diagrams of a FUSC (Female Urine Sampling Container) in accordance with some embodiments of the current invention;

FIG. 2A is a schematic illustration of a top view of a FUSC in accordance with an embodiment of the current invention;

FIG. 2B is a schematic illustration of a side view of a FUSC in accordance with an embodiment of the current invention;

FIG. 2C is a schematic illustration of a FUSC handle, in accordance with an embodiment of the current invention;

FIG. 3A is a schematic illustration of a top view of a FUSC lid with a needle port, in accordance with an embodiment of the current invention;

FIG. 3B is a schematic illustration of a side view of a FUSC lid with a needle port, in accordance with an embodiment of the current invention;

FIG. 4A is a schematic illustration of a top view of a FUSC with a threaded handle fixture, in accordance with an embodiment of the current invention;

FIG. 4B is a schematic illustration of a side view of a FUSC collection body with a threaded handle fixture, in accordance with an embodiment of the current invention;

FIG. 5 is a flow chart illustration of a method of using a FUSC in accordance with an embodiment of the current invention;

FIG. 6A is a simplified pictorial illustration of a perspective side view of a FUSC, in accordance with an embodiment of the current invention;

FIG. 6B is a simplified pictorial illustration of a side view of an open expanded FUSC, in accordance with an embodiment of the current invention;

FIG. 6C is a simplified pictorial illustration of perspective side view of a closed FUSC with a sampling tube attached thereto, in accordance with an embodiment of the current invention;

FIG. 6D is a simplified pictorial illustration of a front view of an open expanded FUSC, in accordance with an embodiment of the current invention; and

FIG. 7 is a flow chart illustration of a method of using a FUSC in accordance with an embodiment of the current invention.

### DETAILED DESCRIPTION OF EXEMPLARY EMBODIMENTS

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention, in some embodiments thereof, relates to a device and method for collecting a liquid, and more particularly but not exclusively, to a container adapted to collecting urine stream samples from a female.

A urine test is a common and important test sometimes used for example in the process of making medical diagnoses. Typically, a container is used by a female to receive a urine sample directly from a urine stream from the user. In many cases, collecting the urine stream raises difficulty and/or discomfort for female users. Some containers that currently exists for use in clinics, hospitals, and/or other settings are not adapted to the anatomical structure of the woman's body. In some cases, a female user has to deal with one or more of difficulties, as described in the following list:
A. There are physical difficulties in taking a sample independently—the female has to widen her legs either while sitting on the toilet or standing, and at the same time hold the container. Maintaining balance during the collection may be difficult, especially for pregnant overweight and/or older women.
B. In the process of giving the sample, which includes holding the container and at the same time giving urine, the woman often fails to direct the urine stream to the opening of the container (for example due to her lack of line of sight and/or inability to see the container) and the urine stream may spill onto her hands, clothes and/or surroundings.
C. In the case of patients (for example, young girls and/or older women) who require assistance in collecting a urine sample, the accompanying assistor, typically the mother or a medical staff, is required to hold the container and put their hands in the toilet to help the patients give the sample for the test and thus be exposed to infections from the toilet and\or urine.
D. The urine sample, as mentioned, should be uncontaminated and sometimes even sterile (for example, in the case of a urine culture test). If the user does not hold the container correctly, the urine sample may come into contact with the user's hand and/or other objects, and the test results may be compromised.
E. When the subject is a young girl the difficulty and discomfort may be even greater for example when the girl's body is small and the plastic container has sharp edges and/or may even scrape or even cut the skin (e.g., on the inner side of the thigh).
F. Urine collection systems that use an intermediate connector between the urine stream and the container may contaminate the urine prior to testing, and may not be properly used by the patient. This may also result in an increase in medical waste.

Some embodiments of the current invention may ameliorate some or all of the above problems and/or solve other problems as will be understood from the disclosure herein.

The present invention, in some embodiments thereof, comprises a female urine sample container, referred to herein by the corresponding acronym FUSC.

Some embodiments of containers of the present invention provide, inter alia, several ergonomic advantages:
A. Physical adaptability—The container is adapted to conform to the anatomy of a female/young girl, with a narrow collection area corresponding to the space between female thighs.
B. The collection area has rounded edges so the subject's skin is not harmed.
C. The lid of the container is easily open
D. The lid of the container turns to a handle.
E. The body of the container is collapsible for example to minimize clinical waste volume and storage/delivery space.
E. The ‘first urine’ is collected to a separated area and is stored separately from the urine collected midstream.
F. Part of the container drainage area is in fluid connection with a vacuum needle mechanism, to which the test tube is configured to connect and collect the midstream urine sample.
G. At the back, there is a raised edge/lip configured to prevent urine from leaking backwards. This raised edge is also useful for guiding the user to position the container at a correct right distance from the urethral area, to prevent sample contamination.

Additionally, some embodiments of containers (FUSCs) of the present invention provide, inter alia, several practical advantages over current-use (prior art) containers, including that they are constructed and configured to enable midstream sample collection, enable intuitive use, are transparent, allow the user a comfortable grip and away from the urine, are lightweight and enable correct positioning thereof without a line of sight.

Additionally, the containers (FUSCs) of the present invention are constructed out of materials suitable for medical/laboratory needs and are easily sealed.

The FUSC is typically constructed and configured to have a shape and/or geometry adapted to collecting urine from a female. For example, a narrow collection area may be narrower at one end, allowing easy insertion between a female's legs, with a less narrow portion at another end allowing for greater volume of storage.

Optionally the FUSC may comprise a handle to allow a user to locate the FUSC in a position to receive the urine stream.

Optionally the handle may enclose removable a vacuum mechanism for transferring the collected urine to a tube or vial.

Optionally, the FUSC may be advantageous over existing urine collection devices due to a minimum of bulky parts. By reducing the number of parts that the collected urine contacts, as it is transferred to a vial and/or test tube, the FUSC may advantageously reduce the possibility of contaminating the urine sample.

The FUSC may be used for temporary urine storage to collect a urine sample from a patient, for example in community clinics and/or a hospital.

### EXEMPLARY EMBODIMENTS

The present invention, in some embodiments thereof, relates to a device and method for collecting a liquid, and more particularly but not exclusively, to a container adapted to collecting urine stream samples from a female.

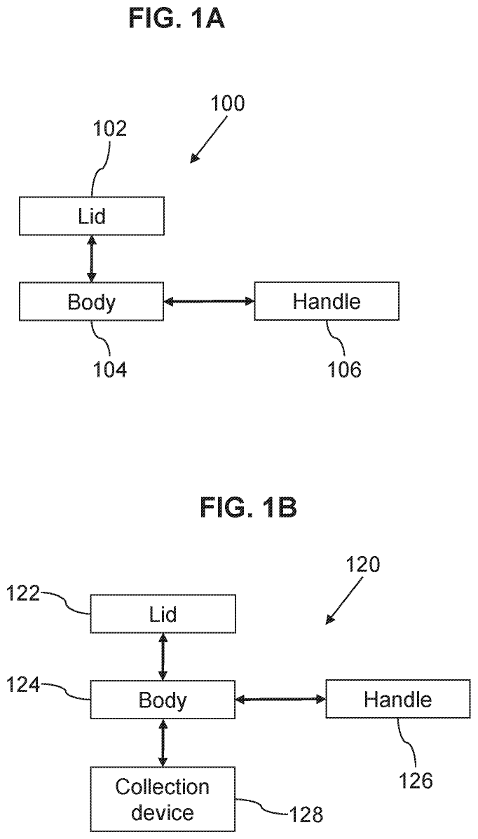

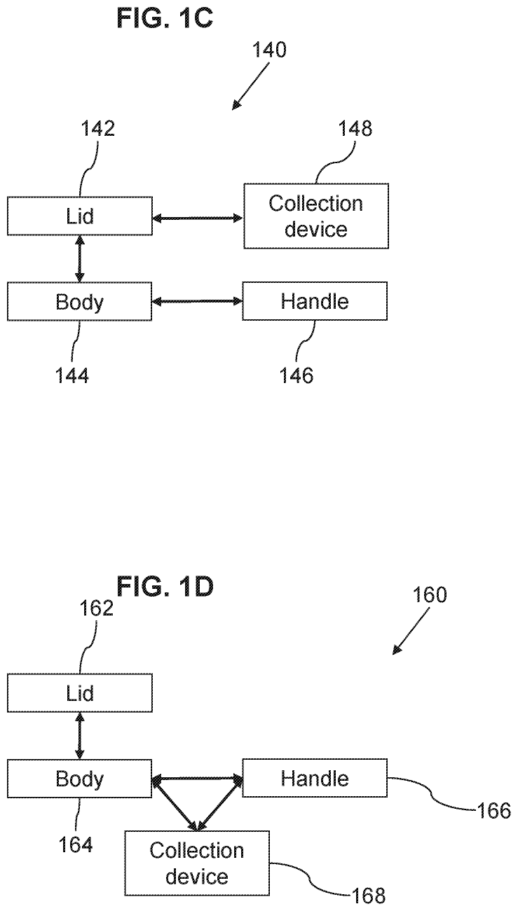

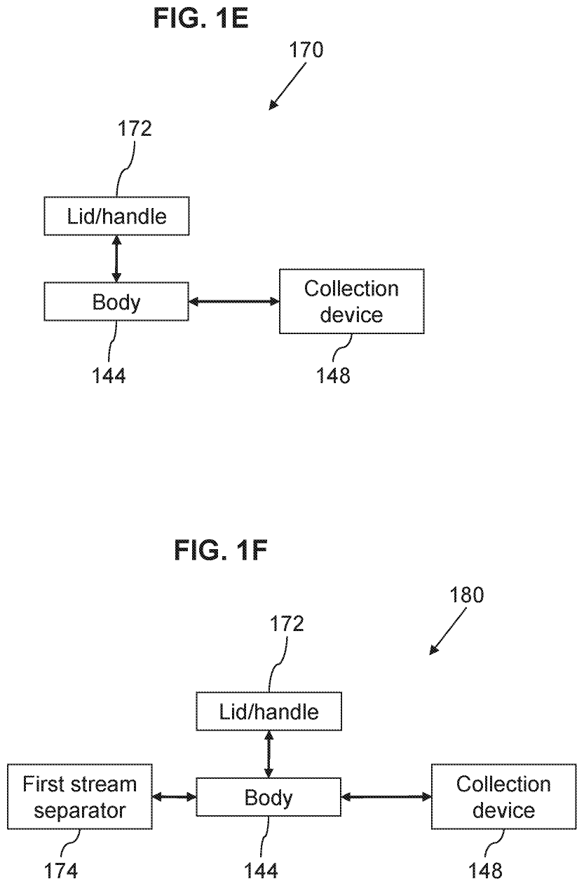

Reference is now made to FIGS. 1A-1F, which are block diagrams of different configurations of FUSC containers 100, 120, 140 and 160, respectively, in accordance with some embodiment of the current invention.

FUSC 100 comprises a lid 102, connectable with a collection body 104, which in turn, is connectable with a handle 106.

Various types of FUSC containers, exemplified by FUSC containers 100, 120, 140 and 160 (e.g., FIGS. 1A, 1B, 1C, 1D, 1E and 1F, respectively), but not limited thereto, may comprise assembly of connectable and/or detachable components comprising a collection body 104, 124, 144, 164, a collection device (e.g., a vacuum device) 128, 148, 168, a handle 106, 126, 146 and 166, and/or a lid 102, 122, 142 and 162 respectively.

Optionally the lid may be removed to allow the FUSC body to collect urine, and the lid may be placed on the body. For example, the lid may prevent the collected urine from spilling from the FUSC.

Optionally the FUSC may constitute only a “sterile intermediate station” between the bladder and further in vitro equipment/apparatus (not shown).

In some embodiments, the handle of the container may be permanently attached thereto. Alternatively or additionally, the container may not include a handle. In some embodiments, the container may include may different kinds of covers and/or may not include a cover. For example, the cover may be built into and/or attached onto the container and/or a handle may extend from the FUSC. For example, the cover may fold out to become a handle in an open position.

In some embodiments (e.g., FUSC 170 of FIG. 1E) a lid 172 may also serve as a handle. For example, the lid may 172 be connected to body 144 such that when the lid 172 is open, it can be easily held to support the body under a woman in a position to collect urine. Various examples, of covers that convert to a handle are possible as illustrated, for example, in the specification below.

In some embodiments, a FUSC (e.g., FUSC 180) may include a first stream separator 174. Optionally, the separator 174, channels a first portion of the urine stream away from a sample compartment. For example, there may be a first stream compartment which receives urine until reaching a predetermined threshold volume [e.g., until the compartment is full] at which point urine is redirected to a sample container. Various examples, of first stream separators are possible as illustrated, for example, in the specification below.

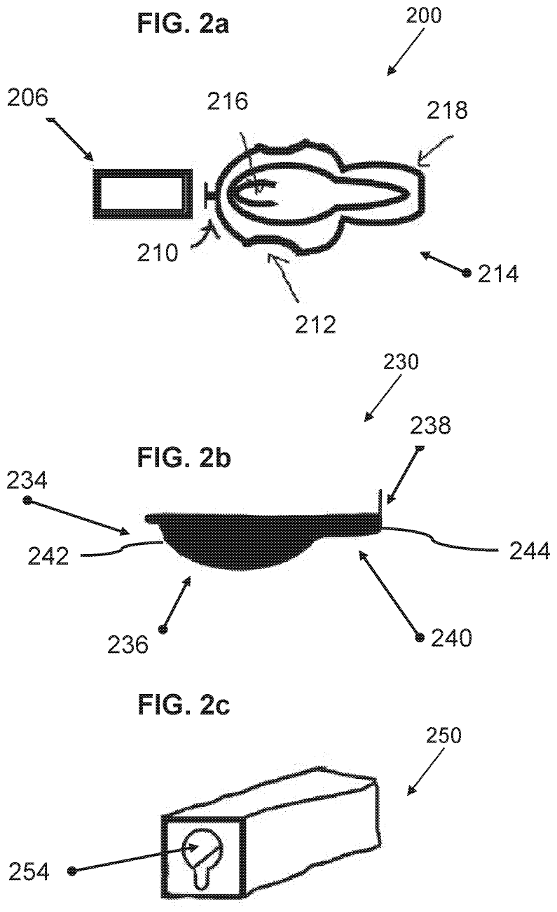

Turning to FIG. 2A, there is seen a schematic illustration of a top view of a FUSC container 200, in accordance with an embodiment of the current invention.

FUSC container 200 may comprise a collection body 214 having a narrow collection area 218, a grip area 212, a drainage area 216, and a handle fixture 210 for affixing a handle 206.

Optionally the one, some, or all components of a FUSC may comprise one or more of the following properties:

1. Made of disinfectable and sterilizable material suitable for medical needs. The collection body, handle, and/or lid may be constructed from any suitable material and/or combination of materials, including plastics, silicon, and/or any other material.

2. Collection body can be easily sealed with the lid.

3. Lightweight.

4. Transparent.

5. A grip area on an opposite end from the narrow collection area.

6. Ease of assembly and use for medical staff

7. Ease of assembly and use for parent/woman.

8. Rounded edges around an upper lip of the collection body where the lid may be attached to the collection body.

Optionally, the container body may comprise a silicon band around at least part of at least one perimeter, for the purpose of better grip. Optionally the lid may comprise a silicon band around at least part of at least one perimeter for better grip.

Optionally the one, some, or all components of a FUSC may provide one or more of the following advantages over the prior art devices and containers:

1. Reduce sample and user's hands contamination.

2. Reduce wrong lab results or diagnostic mistakes.

3. Reduce clinical waste volumes.

4. Reduce TcTi (Time from Complaint to Improvement)

5. Reduce P@C (Time the patient is spending in the clinic)

6. Improve female user experience

7. Improve diagnostic abilities

8. Improve health services provision to female and young girls.

9. Improve self-service for disabled populations

10. Improve financial performance for health provider.

In some embodiments of the present invention, user-friendly apparatus and methods are provided for a female person to collect her own urine sample.

The present invention provides, according to some embodiments, an ergonometric container (FUSC), which is constructed and configured to be drained directly into a vacuum syringe.

Some embodiments of containers (FUSC) of the present invention further include a tactile marker, which assists a woman in placing it properly.

The containers (FUSC) of the present invention are optionally collapsible. For example, a collapsible container may take up less room in medical waste, when disposed after use than rigid containers.

Some embodiments include a first stream repository. Optionally, the first stream repository separates and/or isolates the first stream of urine (which may be contaminated and/or which may be separated from the laboratory sample). The first stream repository optionally includes a one-way valve (e.g., a floating ball as illustrated in an exemplary embodiment).

Container Body

Optionally, the FUSC container body may have a geometry, shape and/or structure adapted to the anatomy of the female comprising a narrow collection area that substantially corresponds to the gap that exists in the space between a female's thighs.

Optionally the collection area may have rounded edges around the edge of the ridge defining the narrow collection area and/or the entire perimeter of the ridge so that the ridge does not scratch the female's skin when placed between the legs.

In some embodiments, the narrow collection area is placed between the female's legs and/or a urine stream is initiated into the FUSC, the urine flow may enter the narrow collecting area and then flow to the drainage area.

Optionally the drainage area may connect the interior to the exterior of the collection body, and may correspond to the shape, dimensions, and/or geometry of a vacuum needle mechanism which may be inserted into the collection body and/or the lid.

The depth of the drainage area may correspond to a vacuum mechanism, for example a test tube which is intended to connect to the collection body. The depth of the drainage area may be 1-2 cm, 2-3 cm, 3-4 cm, 4-5 cm, 5-6 cm, 6-7 cm, 7-8 cm, 8-9 cm, 9-10 cm, 10-11 cm, 11-12 cm, 12-13 cm, 13-14 cm, 14-15 cm, or greater than 15 cm.

Optionally a grip area of the exterior portion of the collection body may be indented, extended, textured, and/or any other way distinguished from the surrounding exterior to and allow easier grip of the collection body.

Optionally the collection body may comprise a handle fixture, for example at an end opposite to the raised lip. The handle fixture may comprise a partial or complete mechanical and/or electro-magnetic means of coupling between a handle and a collection body.

Reference is now made to FIG. 2B, which is a schematic illustration of a side view of a FUSC 230 in accordance with an embodiment of the current invention.

Optionally, at an end of a length of a collection body 234, proximal to a narrow collection area 240 there may be a raised lip or edge 238, extending upwards and away from the collection body, whose function may be to prevent urine from splashing and/or leaking. The raised lip may be used to direct the female user as to how and where to place container 230 at the correct location relative to the urethral area, for example by placing the raised lip at the bottom of her buttocks (not shown), and/or to prevent sample contamination if the container body is too close to the woman's body. Typically, the urine drainage area 236 is at a proximal end 242 of the collection body and the raised lip is at a distal end 244 thereof.

FIG. 2C is a schematic illustration of a FUSC handle 250, in accordance with an embodiment of the current invention.

Optionally, the handle may be detachably attached to the container (200, 230) body via handle fixture 210 (e.g., FIG. 2A). Optionally the handle may comprise a fixture socket 254 at one end for detachably coupling to the handle fixture. Alternatively or additionally, the handle may extend from the FUSC body.

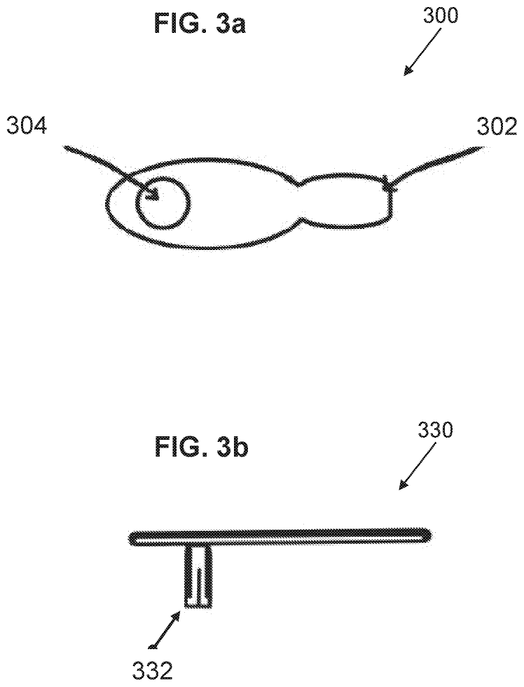

FIG. 3A is a schematic illustration of a top view 300 of a FUSC lid 302 with a needle port 304, in accordance with an embodiment of the current invention.

Optionally the FUSC comprises a lid which may be formed from a flexible, bendable, deformable, and/or soft material. This may include any suitable polymeric material, plastic, rubber, silicone and combinations thereof. Optionally the lid and may cover the container by coupling with the rounded edges of the collection body, for example in a similar manner to plastic food storage boxes.

FIG. 3B is a schematic illustration showing a side view 330 of FUSC lid 302 with a needle port 332 (similar or identical to needle port 304, FIG. 3A), in accordance with an embodiment of the current invention.

Optionally the lid may comprise a needle port in an area corresponding to the drainage area. Optionally the needle port enables an inserted vacuum mechanism to withdraw urine from a sealed FUSC.

Optionally, the needle port may comprise a cavity extending downwards from the lid when placed onto the collection body, for example into the drainage area, a facility for accepting a vacuum mechanism into the needle port, a facility for puncturing a membrane of the vacuum mechanism, for example a protruding needle, thereby enabling a properly inserted vacuum mechanism to withdraw urine from the collection body.

Optionally, a needle and/or vial mechanism may be associated with the lid and/or the body of the FUSC. For example, the mechanism may be disinfectable held to, inserted into, and/or attached to the lid and/or the body. Optionally, association may be in a manner similar to connecting any object to lids of containers for food, medical, urine specimen and or any other purpose.

Optionally, the needle port may comprise means of connecting the negative pressure of a vacuum enabled container, for example a test tube and/or a vial, to the collection body, thereby drawing the urine sample from into the vacuum enabled container.

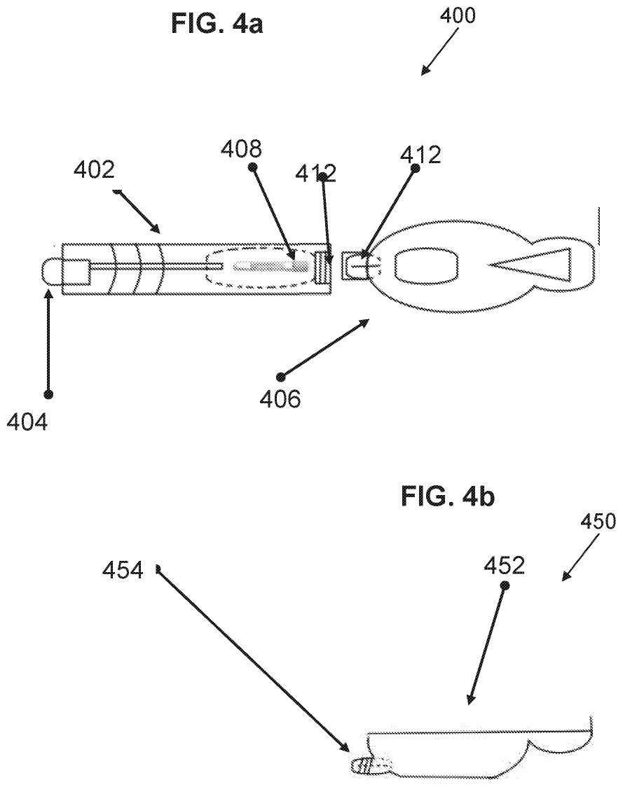

Reference is now made to FIG. 4A, which is a schematic illustration of a top view of a FUSC 400 with a threaded handle fixture 406, in accordance with an embodiment of the current invention.

Optionally, FUSC 400 comprises a handle 402, which may be attached to the collection body by a helical threaded mechanism 410, for example where the handle fixture socket may comprise a threaded port 412. Any male-to-female or female-to-male threading arrangement is within the scope of the present invention. Any other suitable connecting arrangement for the handle and container body is also deemed to be within the scope of the present invention, such as, but not limited to, click-fit, snap-fit, snug-fit, a friction fitting, an interference element and other similar connection alternatives. Additionally or alternatively, the collection body handle fixture may comprise a corresponding threaded extension. The handle may also comprise an actuator 404 for activating the vacuum mechanism 408 (for example an evacuated sample vial). In some embodiments, a cover may fold out and/or extend from the FUSC body. For example, a cover of the FUSC may open to form a handle.

FIG. 4B is a schematic illustration of a side view 450 of a FUSC collection body 452 with a threaded handle fixture 454, in accordance with an embodiment of the current invention.

Optionally the handle may comprise a substantially hollow cavity. Optionally the cavity may accommodate inserting a vacuum mechanism, for example a test tube and/or vial. For example, the vacuum mechanism may be inserted through an opening at one end of the length of the handle.

Optionally the handle fixture and corresponding handle fixture socket may comprise a port communicating between the drainage are of the collection body and the inserted vacuum mechanism. Optionally, the port of the collection body may comprise a pin and/or other mechanism for puncturing a membrane of the vacuum mechanism. Puncturing the membrane may allow vacuum negative pressure to suck collected urine from the collection body into the vacuum mechanism. For example, urine may enter the collection body via a channel within the pin communicating between the exterior end of the pin and the interior of the collection body.

Optionally, the handle may comprise a mechanism for forcing an inserted vacuum mechanism onto the puncture mechanism. In some embodiments, forcing the needle into the vacuum mechanism may puncture the membrane. For example, the handle may comprise an actuator, for example a button and/or extension. Optionally pushing the button transfers the force and/or actuates a device that applies force to push the vacuum mechanism onto the puncture mechanism.

Optionally a force applied to the actuator maintains the sucking of urine by the vacuum mechanism. Optionally, when the force ceases the connection is closed. Alternatively or additionally, the sucking of urine by the vacuum mechanism may continue unaided by any force on the actuator. For example, once the membrane has been punctured the connection and/or suction may continue until action is taken (e.g., disconnecting the collection container).

Optionally the handle may further comprise a latch and/or other holding mechanism to hold the vacuum mechanism in place. For example, holding the mechanism in place may allow continued flow of the urine. Optionally the holding mechanism may be released, for example by applying a force to the actuator and/or any other mechanism.

Optionally the actuator may comprise a mechanism for at least partially withdrawing the vacuum mechanism from the handle

In some embodiments, the FUSC may hold the vacuum mechanism within the handle attached to the collection body. The handle may be hollow to contain the vacuum mechanism. The handle may be screwed onto the collection body, for example by twisting and/or rotating the handle. In some embodiments, screwing the handle to the body of the FUSC may attach then handle and/or the collection mechanism to the FUSC. Alternatively or additionally, the handle may extend from the FUSC body.

Optionally the vacuum mechanism may be mechanically activated. For example, activation may include be pushing the vacuum mechanism against the puncture mechanism. Optionally activation may continue only while the subject will push the actuator. For example, the urine may be transferred from the collection area to the vacuum mechanism only when the actuator is pushed.

Optionally releasing the actuator may allow the vacuum mechanism to retract from the puncture mechanism, for example by means of a spring, thereby ceasing the flow of urine into the vacuum mechanism. Optionally, the vacuum mechanism may be retracted and disconnected from the collection body port by unscrewing or otherwise disconnecting the handle from the collection body.

Optionally the handle may be transparent at least one side or part thereof to allow the user to see when the vacuum mechanism is full.

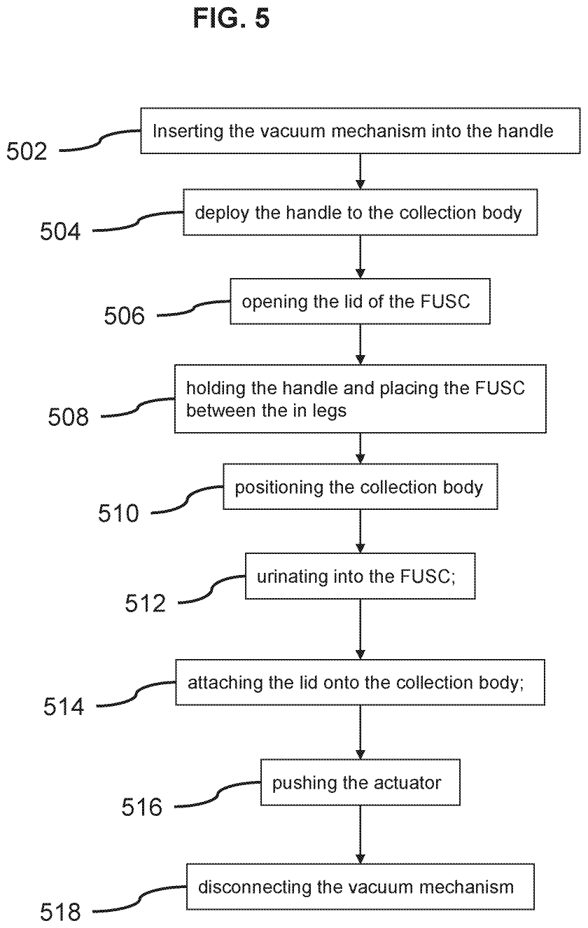

Turning to FIG. 5, there is seen is a flow chart illustrating of a method of using a FUSC in accordance with an embodiment of the current invention.

In some embodiments, the method of using a FUSC may comprise one, some or all the following steps and may use any of the FUSC container embodiments described herein.

First a user, such as a female user inserts a vacuum mechanism into a handle of the FUSC in a vacuum mechanism insertion step 502.

Thereafter, she deploys 504 the handle to the collection body, for example by screwing the handle onto a threaded handle fixture in a handle attaching step and/or by extending the handle from the FUSC body (e.g., by opening a cover to become a handle in the open position).

She then opens the lid of the FUSC, in a lid opening step 506.

In a FUSC placing step 508, the female user holds the handle and places the FUSC between her legs.

In a FUSC positioning step 510, she positions the collection body so that the raised lip is felt on the buttocks and/or another anatomical region of her body.

She then passes a urine sample in a urinating into the FUSC step 512. Once this is completed, she attaches the lid onto the collection body in a lid attaching step 514. The full urine sample has now been collected in the FUSC, including the initial (first and mid-stream sample).

The female user then pushes the actuator on the handle with sufficient force to puncture the membrane allowing the urine to transfer to a vacuum mechanism, in a pushing actuator step 516.

Finally, she disconnects the vacuum mechanism from collection body, for example by unscrewing the handle or otherwise disconnecting the handle from the collection body in a disconnecting vacuum mechanism step 518.

Optionally the method may comprise using a FUSC where the vacuum mechanism is inserted into a needle port. Optionally, the needle port may be in the lid and/or elsewhere (e.g., on the body of the FUSC). For example, inserting the vacuum mechanism into the needle port may actuate the transfer of urine.

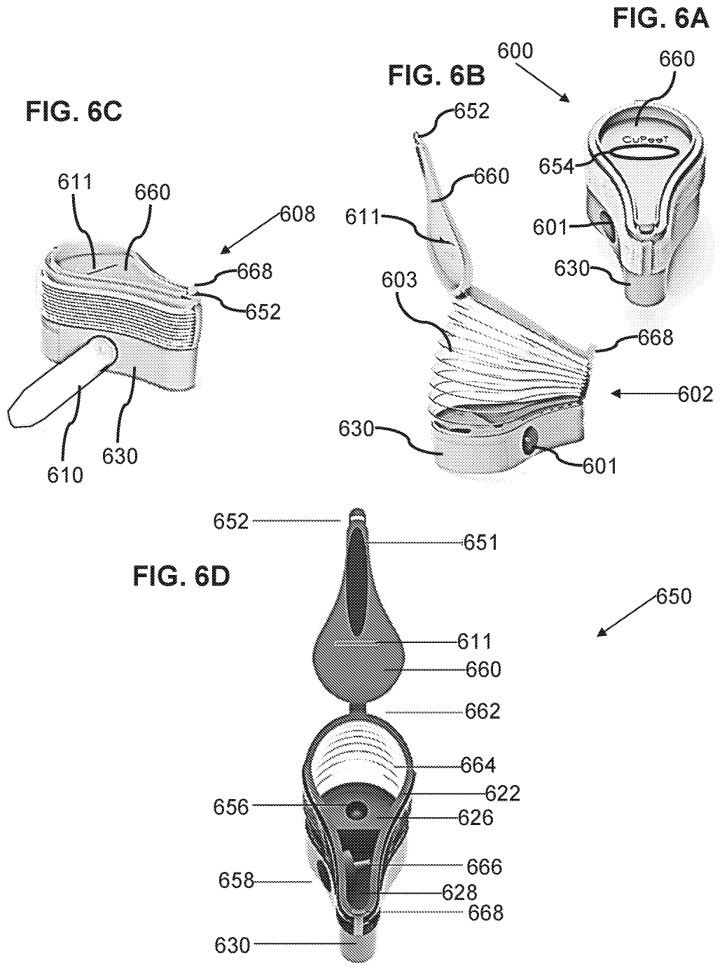

Some non-limiting pictorial illustrations of the FUSCs of the present invention are shown in FIGS. 6A-6F. The FUSCs of the present invention are constructed and configured to enable a female user, who is wearing heavy clothing, such as wearing a long skirt, to perform a urine test without seeing the container and without soiling her clothes.

Reference is now made to FIG. 6A, which is a simplified pictorial illustration of a perspective side view of a FUSC 600, in accordance with an embodiment of the current invention. As can be seen, FUSC 600 has a saddle-like shape, when in a closed position. It also comprises a port or recess 601 for receiving a tube or vial 610 (e.g., FIG. 6C). FIG. 6B shows a side view of an open expanded FUSC 602, in accordance with an embodiment of the current invention. The open expanded FUSC comprises an expandable section 603. For example, the expandable section may be made of an elastic material (e.g., rubber or silicone) and/or may include a folding section (for example, section 603 includes a wall with back-and-forth folds like an accordion). In some embodiments, the expandable section may be biased to an expanded form and/or may be designed to stand stability on a surface with fluid contents. Alternatively or additionally, the expandable section may be biased to a folded form. Optionally, there may be a support that can hold the section in an expanded state. For example, the device of FIGS. 6A-6D includes a saddle shaped top frame and base and/or a folding wall that expands the space between the base and the frame.

The FUSCs of the present invention may have a length ranging between 2 to 10 cm and/or 10 to 20 cm and/or 20 to 30 cm. A front portion of the FUSC may have a width ranging between 0.5 to 2 cm and/or 2 to 5 cm and/or 5 to 10 cm and/or 10 to 20 cm. A rear portion of the FUSC may have a width ranging between 0.5 to 2 cm and/or 2 to 5 cm and/or 5 to 10 cm and/or 10 to 20 cm. When opened the FUSC may be of a depth ranging between 0.5 to 2 cm and/or 2 to 5 cm and/or between 5 to 10 cm and/or between 10 to 25 cm. The weight of FUSC may range between 2-10 grams and/or 10 to 20 grams and/or 20 to 60 gram and/or 40 to 80 grams, when empty. The FUSC may receive a volume of urine of between 1 to 10 mL and/or 10 to 20 mL and/or 20 to 40 mL and/or 50 to 100 ml and/or 100 to 200 ml and/or between 200 to 400 ml and/or between 400 to 800 ml and/or any other suitable size.

Turning to FIG. 6C, there is seen a perspective side view of a closed FUSC 608 with a sampling tube/vial 610 attached thereto, in accordance with an embodiment of the current invention. When the FUSC is closed, its volume is reduced by 30%, 40%, 50%, 60%, 70% or more. This is effective in reducing the volume of medical waste.

In some embodiments, a lid 660 may open into a handle for holding the FUSC 608. For example, the lid 660 may be connected the FUSC 650 such that when the lid 660 is opened in project past the front of the chamber of the FUSC 650 and has limited movement with respect to the chamber such that holding the open cover 660 limits movement and/or holds the FUSC 650. For example, lid 660 is connected to the front of the FUSC 650 by a hinge (e.g., the hinge may include a pin around with the cover 660 rotates and/or a flexible connection that allows limited movement of the cover 660 with respect to the body of the FUSC 650. For example, the angle of rotation between the cover 660 and the body of the FUSC 650 may be limited such that holding the cover 660 supports the body of the FUSC.

FIG. 6D shows a non-limiting simplified pictorial illustration of a front view of an open expanded FUSC 650, in accordance with an embodiment of the current invention. The FUSC may comprise one or more or all of the following features/items: an opening tab 652 and ergonomic handle/lid 660, a pH stick opening 611 to enable the introduction of a pH dipstick or other dipstick (such as, but not limited to glucose, ketones, proteins, pregnancy hormones and the like). The FUSC may further comprise any one or more of the following, a mid-stream separation mechanism 656 for separating initial urine passed from mid-stream urine, a connector to lab apparatus (in vitro connection) 658, a stabilizer axis 662, an accordion-like expanded wall section 664 (like wall section 603), which when pressed together size-reduces/compacts the FUSC, a vacuum mechanism 666 and a position marker 668. The female user places place the position marker on her lower buttock's cheeks. The FUSCs of the present invention are constructed and configured to enable a female user, who is wearing awkward and/or heavy clothing (such clothing may be common in very religious women, for example, long skirts for Jewish Haredi women and/or hijabs Muslim women, especially in the presence of any male outside her home and/or Tudong/Tudung for Indonesian woman and/or a Chador). The problem may occur for example when the is a heavy cover over the woman's body, making it difficult to perform a urine test without seeing the container and without soiling her clothes.

FIG. 6D shows a simplified pictorial illustration of perspective side view of an open FUSC 650, in accordance with an embodiment of the current invention. A FUSC may contain some or all of the following features:
a. An opening tab 652, configured to enable a user to hold the tab to lift a lid 660 and open the container before use. This tab may also be used to close the lid after the urine has been collected in the container. The tab may optionally be fitted to mate with a position marker 668. For example, the position marker 668 may be an interference element that mates with tab 652
b. In some cases, a medical professional, such as a nurse will need to perform an immediate test on the urine, such as pH or glucose. This test may be performed using a dipstick and/or a slot (e.g., a pH stick slot 611).
c. The container comprises a mid-stream separation mechanism 656. For example, there is a closed first stream compartment 626 near the expected first contact point with the urine (e.g., the front of the FUSC 650 and/or there is a collector channel (e.g., the front wall is designed to channel and/or funnel the urine stream towards the first stream chamber). Optionally, there may be a one-way valve and/or a cut off valve (e.g., a ball valve). The first volume of urine falls on the open ball valve into a first stream compartment 626. When a predefined volume (e.g., a threshold volume) enters the first stream compartment 626 the ball valve closes (e.g., due to buoyancy). Thereafter, urine is directed into a sample compartment 628.
   The first stream compartment 626 may hold a volume ranging between 0.5 to 1 mL and/or 1-2 mL and/or 2 to 4 ml and/or 4 to 10 mL and/or 10 to 20 mL and/or 20 to 40 mL and/or 40 to 100 mL and/or 100 to 200 mL and/or any other suitable volume.
   The sample compartment may hold a volume ranging between ranging between 0.5 to 1 mL and/or 1-2 mL and/or 2 to 4 ml and/or 4 to 10 mL and/or 10 to 20 mL and/or 20 to 40 mL and/or 40 to 100 mL and/or 100 to 200 mL and/or 200 to 400 mL and/or 400 to 800 mL and/or any other suitable volume.
d. An in vitro connector 658—the connector is optionally configured to place a vacuum attachment (not shown) into the hollow and press it against an inner needle (not shown). A vacuum mechanism 666 sucks the urine from the sample compartment to the in vitro connector. In some embodiments, the vacuum is more effective when the lid is closed and/or ineffective when the lid is open. Alternatively or additionally, the vacuum is more effective when the lid is open and/or ineffective when the lid is closed.
e. A sanitary sticker 651 disposed on an inner side of a lid 660. The lid may serve as a handle. The sticker is to be removed by the user after complete urination and before closing the lid. This sticker is configured to help to preserve sanitation of the inner side of the lid, and helps keep the sample uncontaminated.
f. An ergonomic (convenient) handle 651—a long part of the lid may be used as the handle. The female user grips the handle to put the container in position before delivering a urine sample. The handle/lid may comprise a thumb grip, similar or identical to grip area 212 (e.g., FIG. 2A).
g. A hinge. For example, the hinge 662 may include a flexible strip bends on one plane allowing movement of the lid rotating around an axis but is stiff in another plane preventing twisting of the body of the FUSC with respect to the cover. For example, the hinge 662 may include a flexible plastic ribbon (e.g., of the same material as the cover 651) which bends along the same plane as the cover 660 and/or allows movement of the cover upward, downward, forward and/or backward by inhibits movement sideways and/or rotation around an axis running from the front to the back of the body of the FUSC. This mechanism is used for stabilizing the container while the female user is filling it with the urine sample. The axis provides support to the handle and further enables the lid/handle to be opened at an angle of up to 270 degrees, up to 210 degrees, up to 180 degrees 120 degrees, 90 degrees (e.g., see FIGS. 6C and 6D) relative to the closed position (e.g., FIG. 6A).
h. A size reduction mechanism 664—the body of FUSC 650 is optionally made of elastic material which is expandable and/or flexible. This material may comprise plastic or bio plastic, which is disposable and degradable. The size reduction mechanism may comprise a spring mechanism or other means to spring open when the opening tab is released/opened. Additionally or alternatively, a bag or other receptacle (not shown) may line the container.
i. A vacuum mechanism 666, such as a covered needle, which is used to transfer the urine from the sample compartment to the in vitro testing vessel.
j. A position marker 668—since the female user has no eye contact with the container, she a tactile marker may be supplied to help her properly place the FUSC to catch urine. For example, the position marker may be configured so that when she places the position marker on her lower buttock's cheeks, the FUSC is properly placed to receive urine.
k. The FUSC further comprises a soft upper lip or rim 622, this is designed to prevent the user being scratched or otherwise injured.

Typically, lid 660 and a base 630 of the FUSC are constructed out of a rigid material, such as a rigid plastic and may contain weights (not shown) for extra stability.

The FUSC may comprise a pH stick slot 611 (e.g., FIG. 6D). Optionally, the slot 611 is positioned near the sample compartment and/or configured such that a stick passed through the slot will enter the sample chamber. Optionally, the slot is in the cover of the FUSC.—In some cases, a medical professional, such as a nurse will need to perform an immediate test on the urine, such as pH or glucose. This test may be performed using a dipstick. The slot allows the nurse to place the stick in the urine container. The FUSC further comprises a pH slot sticker 654 (e.g., see FIG. 6A), which is removed in some case by a medical professional and in others by the user, before using the pH stick (not shown). In some cases, removal of the sticker will release the vacuum mechanism.

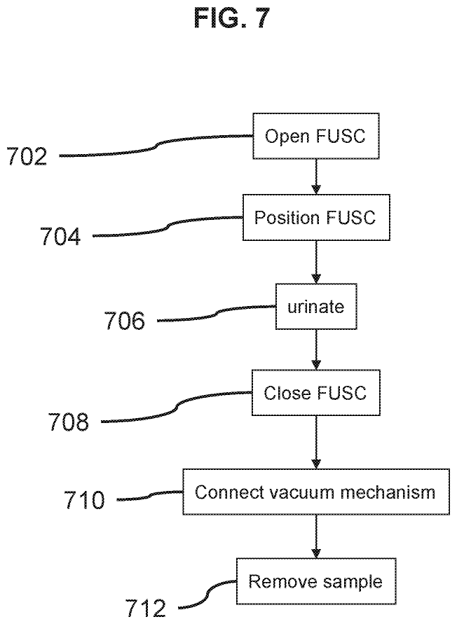

Reference is now made to FIG. 7, which is a flow chart illustration of a method of using a FUSC in accordance with an embodiment of the current invention. Embodiments of the invention may use one, some or all of the steps:

Typically, the female user first receives the FUSC at a clinic. It is typically closed and shrunk to a minimum size. The user takes it to the toilet.

In an opening FUSC step 702, the user opens/retracts the upper lid (such as by pulling opening tab 652 (e.g., FIG. 6D) to its fully expanded in size, using one hand and holds bottom with the other hand. She may collect the dress/skirt in her other hand.

In a FUSC positioning step 704 she uses position marker 668 to position the FUSC. For example, the marker may be positioned at the base of her buttocks.

In a providing a urine sample step 706, the user provides a urine sample into the FUSC and may count for a given time, such as, but not limited to 5 seconds.

According to some scenarios, the user removes the FUSC from urine stream and completes urination in the toilet. This may not always be necessary.

If required, she then releases her dress/skirt from one hand, still holding the FUSC with the other hand.

In a FUSC closing step 708, the user holds the FUSC with one hand and optionally releases sanitary sticker 651 with the other hand and then closes the lid.

In an in vitro element inserting step 710, the user inserts an in vitro element to connector 658 the vacuum mechanism.

In a remove in vitro sample step 712, a sample of the urine from the sample compartment to the in vitro equipment (not shown) and shrinks back the FUSC to its minimal volume with the size reduction mechanism collapsed.

The FUSC is now ready for disposal to trash.

It is expected that during the life of a patent maturing from this application many relevant technologies will be developed and the scope of the terms tube, vial, vacuum mechanism, actuator, lid, handle, puncture mechanism, connector and/or plastic is intended to include all such new technologies a priori.

In sum, although various example embodiments have been described in considerable detail, variations and modifications thereof and other embodiments are possible. Therefore, the spirit and scope of the appended claims is not limited to the description of the embodiments contained herein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Optionally the therapy may also be stopped by instructions from a human.

As used herein the term “about” refers to ±5%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A device enabling a female user to collect a urine sample, the device comprising:
a sample compartment for collecting a sample of the urine; and
a first stream separator for separating a first stream of urine from the sample of the urine and directed the sample of the urine to the sample compartment.

2. The device of claim 1, further comprising a lid for closing the sample compartment after the urine sample has been received.

3. The device of claim 2, wherein said lid is movable connected to said device such that when open the lid projects from an anterior edge of the device and is configured for use as a handle holding the device in a position to receive a urine stream.

4. The device of claim 1, wherein the sample compartment and the first stream separator are included in a urine collection body having a saddle shape.

5. The device of claim 4, wherein said body comprises a wider collection area and a narrower drainage area.

6. The device of claim 1, wherein the sample compartment is expandable.

7. The device of claim 6, wherein the sample compartment includes an accordion-like expandable wall.

8. The device of claim 1, further comprising a tactile position marker.

9. The device of claim 4, wherein said collection body includes a lip configured to contract the body of the user and wherein said tactile position marker includes a protrusion from the lip.

10. The device of claim 9, wherein the body has a wide proximal end configured to face anteriorly during use and a distal end configured to fit between legs of the user and face posteriorly and wherein the tactile position marker is disposed at a distal portion of the body.

11. The device of claim 1, wherein the first stream separator comprises a first stream compartment for storing said first stream.

12. The device of claim 11, wherein said first stream compartment includes a cut off valve for closing said first stream compartment after receiving a predetermined quantity of fluid.

13. The device of claim 1, further comprising a needle port opening into said sample compartment for withdrawing at least a part of said urine sample under vacuum.

14. The device of claim 13, further comprising a tube receiving port for receiving at least one of a vial and a tube.

15. The device of claim 1, further comprising at least one of:
a. an opening tab;
b. a dipstick port;
c. a connector to lab apparatus (in vitro connection),
d. a stabilizer axis;
e. a vacuum mechanism;
f. a sanitary sticker; and
g. a position marker.

16. A method collecting a urine sample from a female user, the method comprising:
providing a device including a sample compartment and a first stream separator;
directing a stream of urine from said user into said device;
separating a first part of said stream of urine and directing it away form said sample compartment and
collecting a volume of said urine stream subsequent to said first stream into said sample compartment.

17. The method of claim 16, further comprising retaining a sterility of said volume of urine.

18. The method of claim 16, further comprising transferring said volume directly from said sample compartment to a sample tube.

19. The method of claim 16, wherein said method is performed by said female user.

20. The method of claim 16, further comprising:
a. opening a lid of the device to an open position;
b. holding said device with said lid in said open position during said directing; and
c. closing said lid after said directing to seal said sample compartment.

21. A device for collecting a urine sample from a female subject comprising:
a saddle-shaped body including
an expandable sample compartment configured expand to receive a urine sample;
a vacuum extractor port opening to said sample compartment and configured for draining said urine sample directly from said sample compartment to a vacuum sampler.

22. The device of claim 21, wherein said saddle shaped container comprises a wider anterior collection portion and a narrow posterior drainage portion.
